(19) United States
(12) Patent Application Publication
(10) Pub. No.: US 2009/0270835 A1
(43) Pub. Date: Oct. 29, 2009

(54) STEERABLE SONOHYSTEROGRAPHIC INJECTION CATHETER FOR UTERINE CANCER SENTINEL LYMPH NODE MAPPING

(76) Inventor: David M. Kushner, Middleton, WI (US)

Correspondence Address: WISCONSIN ALUMNI RESEARCH FOUNDATION, C/O BOYLE FREDRICKSON S.C, 840 North Plankinton Avenue, Milwaukee, WI, 53203, US

(21) Appl. No.: 12/108,547

(22) Filed: Apr. 24, 2008

(51) Int. Cl. A61M 31/00

(52) U.S. Cl. 604/515; 604/523

(57) ABSTRACT

A steerable sonohysterographic injection catheter for identifying sentinel lymph nodes in uterine cancer patients includes a tubular shaft housing a needle extendible therefrom. The needle is preferably sized for intrauterine insertion and configured to deliver a fluid such as a contrast media to a tumor location. The catheter preferably includes a sealing device such as a selectively inflatable balloon disposed at a position along the outside of the catheter. The sealing device is configured for placement at an opening of a patient's cervix to prevent the leaking of the contrast media out of the uterus through the cervix. The distal end of the catheter is steerable so as to allow the tip of the needle to be positioned substantially near the tumor location.

### CROSS-REFERENCE TO RELATED APPLICATIONS

None

### STATEMENT REGARDING FEDERALLY SPONSORED RESEARCH OR DEVELOPMENT

None

### BACKGROUND OF THE INVENTION

The present invention relates to sonohysterography and in particular to a steerable sonohysterographic injection catheter for cancer sentinel lymph node mapping.

During treatment for certain forms of cancer, such as uterine cancer, for example, cancerous tumors are surgically removed along with sentinel lymph nodes. Sentinel lymph nodes are the first lymph nodes linked to the site of the cancer. Sentinel lymph nodes are identified by determining which lymph node is first to filter fluid generated by the cancer. This determination can be done in a variety of different ways including injecting dye into the affected area and identifying the lymph node where the dye first begins to collect. Removal of these sentinel lymph nodes can help to prevent the spread of the cancer to the lymph duct system thereby stemming the progression of the cancer.

In treating uterine cancer, it is often difficult to inject the dye into the uterus close enough to the tumor to identify the sentinel lymph nodes. Accordingly, physicians may remove all lymph nodes associated with the uterus in hopes of stemming the spread of the cancer. However, it can be difficult for physicians to properly locate all of the lymph nodes associated with the uterus due to a variety of factors such as, for example, patient obesity. Or a full lymphadenectomy may be inadvisable because of risk factors such as hypertension or diabetes. Further, full lymphadenectomy can increase patient morbidity, operative time, incision size, blood loss and hospital stay length.

Some physicians have attempted to solve the aforementioned problem by injecting the dye through the patient's abdomen through the uterine wall and into the uterus in order to track the fluid flow into the lymph system. However, this particular procedure is not very accurate as it is difficult to inject the dye close to the base of the tumor. Further, the injected dye may leak from the uterus into the abdomen and outside of the lymph node system and carry cancer cells into the abdomen thereby promoting the spread of the disease.

Another method of identifying sentinel lymph nodes that has been employed is the use of hysteroscopic injection to introduce the dye to the uterus. However, hysteroscopic injection has been known to cause both fluid and viable cancer cells to spill out of the fallopian tubes and into the abdominal cavity.

### BRIEF SUMMARY OF THE INVENTION

The present inventor has recognized that sentinel lymph node mapping in uterine cancer patients may be performed utilizing a steerable sonohysterographic injection catheter. This catheter permits the injection of tracer materials about the tumor without substantial distention of the uterus with fluid such as might promote transfer of cancer cells out of the fallopian tubes into the abdomen.

Specifically, the present invention provides a steerable sonohysterographic injection catheter comprising a needle housed in a tubular shaft. The needle is extendible from the tubular shaft for intrauterine insertion and configured for delivery of a fluid therethrough. In addition, a distal end of the needle is selectively steerable by a user to allow the user to position the distal end of the needle near a tumor location. Finally, a sealing device is disposed along the catheter and positioned to prevent fluid from leaking out of the patient's uterus through the patient's cervix.

It is thus one object of an embodiment of the invention to provide a catheter for mapping sentinel lymph nodes that is capable of injecting dye around the tumor without substantial distention of the uterus with liquid such as might seed tumor cells to the abdomen.

The tubular shaft may comprise a multi-layer structure comprising a rigid outer sheath and a relatively flexible inner sheath. In addition, a control member may be provided coupled to a side of the inner sheath and configured to pull the side of the inner sheath thereby causing the inner sheath and the needle housed therein to bend.

It is thus one object of one embodiment of the present invention to provide a mechanism that may work with a standard hypodermic needle to provide controllable flex to the needle.

The catheter of the present invention may further include a handle disposed at one end thereof and operably coupled to a control member to operate the control member.

It is thus one object of one embodiment of the present invention to provide a handle that retains the control member for ready access by the physician.

The catheter of the present invention may further comprise a first port disposed in the handle for receiving the needle therethrough and positioning the needle in the tubular shaft. Further, a proximal end of the needle may be coupled to a syringe for receiving fluid therefrom.

It is thus another object of one embodiment of the present invention to collect the controls and ports at a single location for better management by the physician.

The sealing device of the catheter may comprise a balloon in communication with a second port configured to deliver an inflation liquid to the balloon for selective inflation thereof.

It is thus another object of one embodiment of the present invention to provide a selectively inflatable sealing device for preventing leakage of fluid from the uterus through the opening of the cervix suitable for a variety of situations.

The needle of the catheter may be substantially flexible and configured to assume a predetermined curvature upon extension from the tubular shaft.

It is yet another object of the present invention to provide an extremely simple mechanism providing controllable curvature simply by changing the insertion depth of the needle.

The needle of the catheter may be composed of a memory alloy such as, for example, nickel titanium.

Thus it is another object of the present invention to provide a needle that assumes its curvature based on the properties of memory alloys.

The inner sheath of the catheter may be configured to assume a preferred radius of curvature upon extension from the outer sheath.

It is thus another object of the present invention to provide a flexible inner sheath that eliminates the need for a separate control member while allowing the use of standard hypodermic needles.

The inner sheath may be composed of a memory alloy configured to automatically assume the preferred radius of curvature upon extension from the outer sheath.

It is thus yet another object of the present invention to provide an inner sheath that may flex based on the properties of a memory alloy.

In another embodiment of the present invention, a method of identifying sentinel nodes associated with uterine cancer first requires the insertion of a catheter into a patient's uterus. Next, a liquid is introduced through the catheter to expand the walls of the uterus while still preserving the relative closure of the fallopian tubes. A distal end of the catheter is then steered to a point near a tumor located in the patient's uterus. A hollow needle is then inserted through the catheter and guided to the tumor location. A tracking material is then injected through the needle at a position relatively near the tumor location to allow for the observing of the tracking material as it accumulates in the patient's lymph system thereby allowing for the identification of a sentinel node associated with the cancer.

It is thus another object of the present embodiment to provide a method of identifying sentinel nodes associated with uterine cancer with reduced risk of seeding tumor cells to the abdomen.

The method of identifying sentinel nodes associated with uterine cancer of the present invention further may include inserting plugs into the fallopian tubes.

It is thus another object of the present embodiment to further reduce fluid leaking out of a patient's fallopian tubes so as to prevent the carriage of cancer cells to the abdominal cavity.

The method of the present invention further requires less than 200 mL of liquid to be injected into the uterine cavity.

It is thus yet another object of one embodiment of the present invention to provide a method that requires a relatively small amount of liquid for performance thereof, thereby decreasing the likelihood that fluid will leak out of the uterine cavity and carry cancer cells with it.

These particular objects and advantages may apply to only some embodiments falling within the claims, and thus do not define the scope of the invention.

### BRIEF DESCRIPTION OF THE DRAWINGS

FIG. 1 is a perspective view of a first embodiment of a catheter of the present invention;

FIG. 2 is a fragmentary perspective cut-away of a distal end of the catheter of the first embodiment of the present invention;

FIG. 3 is a partial sectional elevation view of a portion of the catheter of FIG. 1 taken along lines 3-3 showing the catheter retracted for insertion in the uterus;

FIG. 4 is a partial sectional elevation view of a portion of the catheter of FIG. 1 taken along lines 4-4;

FIG. 5 is an elevation view of an end of the catheter of FIG. 3 taken generally along lines 5-5;

FIG. 6 is a perspective view of a distal portion of a catheter of a second embodiment of the present invention;

FIG. 7 is a perspective view of a distal portion of a catheter of a third embodiment of the present invention;

FIG. 8 is plan view of the catheter of the present embodiment in position within the uterus; and

FIG. 9 is a schematic representation of a patient's lymphatic system showing the sentinel nodes.

### DETAILED DESCRIPTION OF THE INVENTION

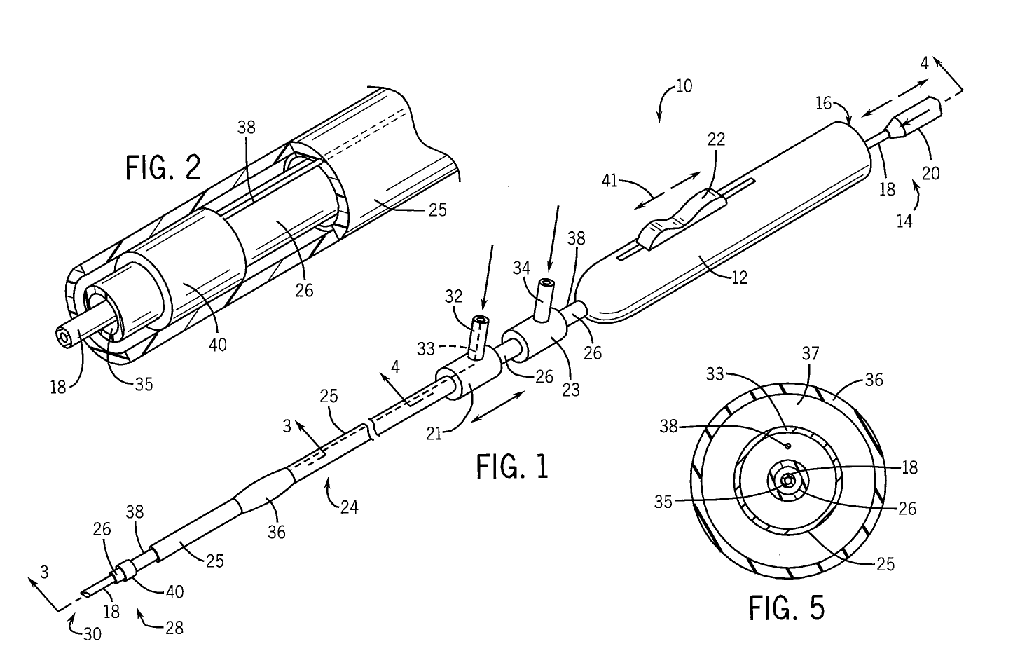

Referring now to FIGS. 1-5, and initially to FIG. 1, a first embodiment of a steerable sonohysterographic injection catheter 10 of the present invention includes a handle 12 at a proximal end 14 of the catheter 10. The handle 12 includes a first port 16 for receiving an injection needle 18 therethrough. Injection needle 18 is sized for intrauterine insertion and includes a proximal end configured to be in communication with a syringe 20 or other such fluid delivery device. Handle 12 further includes a thumb slide 22 disposed on a surface thereof and in communication with an inner sheath as will be described for control of angular motion of the needle 18.

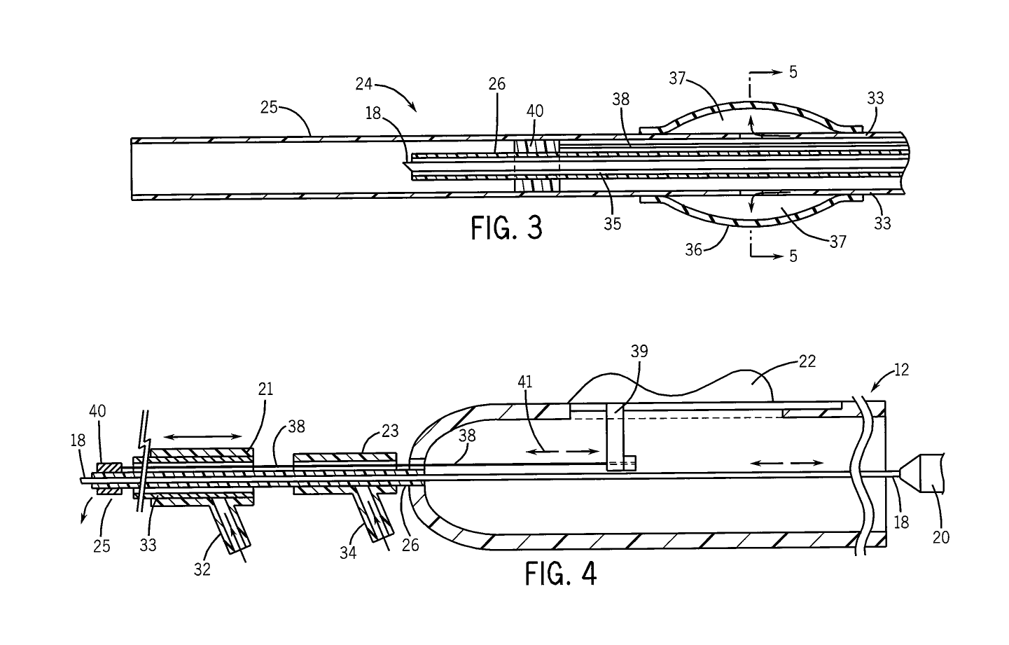

Referring also to FIGS. 3 and 5, catheter 10 includes a multi-layer probe 24 having an outer rigid tubular sheath 25 fabricated of stainless steel or the like and an inner sheath 26 housed within the outer sheath 25 and configured for telescopic extension therefrom. The outer sheath 25 may have a diameter of approximately five millimeters to be substantially smaller than an endoscope and require little distention of the uterus. Inner sheath 26 may be extended from outer sheath 25 by sliding either a first collar 23, or the handle 12, attached to the inner sheath 26, with respect to a second collar 21 attached to the outer sheath 25. Injection needle 18 extends longitudinally through the inner sheath 26 for extension from a distal end 28 of the inner sheath 26 and thus the catheter 10. Injection needle 18 may be extended from the inner sheath 26 by movement of the syringe 20 with respect to the handle 12 or collar 23. The distal end 30 of needle 18 is generally steerable by changing an extension of the inner sheath 26 through the outer sheath 25, a rotation of the inner sheath 26 through the outer sheath 25, and/or an adjustment in angle of the inner sheath 26 with respect to the outer sheath 25. These adjustments allow the user to position the distal end 30 of needle 18 near different tumor locations to allow for mapping of the sentinel lymph nodes associated with the tumor as will be discussed further below.

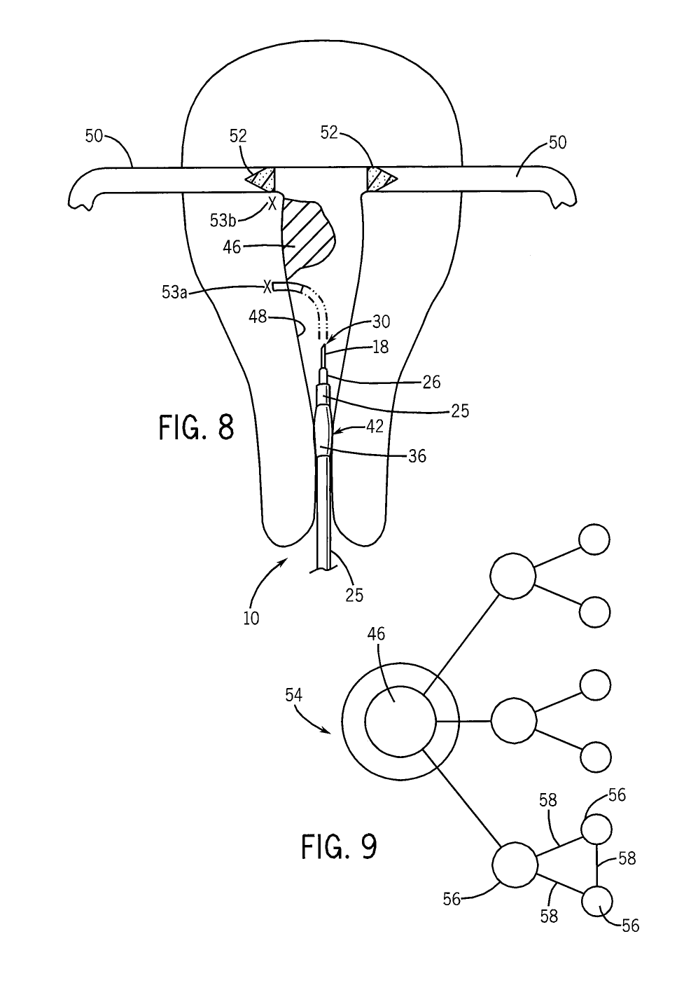

Referring to FIGS. 1, 3, and 5, second port 32 forming part of collar 21 of probe 24 is configured to be in communication with a sealing device, such as a balloon 36, by way of a channel 33 formed in outer sheath 25. Balloon 36 may be inflated with a liquid, for example, saline, thereby creating an inflated portion 37 for sealing the opening of the cervix. As such, saline or another such fluid may be injected through second port 32 for travel through channel 33 so as to selectively inflate balloon 36. Referring momentarily to FIG. 8, the balloon 36 is preferably positioned at a point along the probe 24 such that during operation, the sealing device is positioned at the opening of a patient's cervix. Accordingly, the balloon 36 may be selectively inflated by a user to create a seal at the opening of the cervix to reduce the amount of injected liquid, such as saline or a contrast medium, from leaking out of the patient's uterus around the catheter 10 while inner sheath 26 is telescopically extendible from outer sheath 25 for mapping of the sentinel lymph nodes. Once inflated, the balloon 36 holds the outer sheath 25 essentially stationary.

Referring now to FIGS. 1, 4 and 5, third port 34 is configured to be in communication with the distal end 30 of catheter 10 through a channel 35 formed between the inner sheath 26 and the needle 18. Fluid, such as saline, may be injected through third port 34 for travel through channel 35 to fill the uterine cavity for the purpose of expanding the otherwise collapsed uterine walls in order to create space for the performance of the sonohysterography. The distal end of channel 35 may be sealed within an O-ring or the like (not shown). Once the uterus is sufficiently distended, a contrast medium or dye such as T-99 radiocolloid may be injected by syringe 20 through injection needle 18 for delivery to the uterus to facilitate the sentinel lymph node mapping.

Referring now to FIGS. 1, 2 and 4, the first embodiment of the present invention includes a control member such as wire 38 operably coupled between the distal end of inner sheath 26 and thumb slide 22. As can be best seen in FIG. 3, wire 38 attaches to the inner sheath 26 at its distal end through an internal collar 40 forming a coupling member that holds the wire 38 offset from an outer wall of the inner sheath 26. Wire 38 preferably comprises a relatively thin metal or plastic filament or similar such structure; however, it is understood that any number of similar such structures, strong in tension or compression, may be capable of performing the required operation.

In operation in the first embodiment of the present invention, the user manually adjusts the curvature of the inner sheath 26. As shown in FIG. 4, thumb slide 22 may move internal arm 39 longitudinally as indicated by arrow 41 to pull wire 38 along a longitudinal path represented by arrows 41. The wire 38 displaces a side of the inner sheath 26 thereby causing a curvature of inner sheath 26 through a range of angles according to the motion of the thumb slide 22. Accordingly, the needle 18 housed in inner sheath 26 is effectively curved therewith against the natural resilience of the inner sheath 26. In this way, the operator of the catheter 10 can manually adjust the curvature of the needle 18 of the catheter 10 of the present invention for fine control over the curvature thereof at any point of extension of the inner sheath 26 into the uterus. As such, the operator can better position the distal end 30 of the needle 18 near a tumor location in a patient's uterus. While the wire 38 is preferably used in tension to pull on a side of the inner sheath 26, it will be understood that a suitably stiff wire may also operate in compression to push away one side of the inner sheath 26 to promote curvature. The curvature of the needle 18 is independent from the rotation of the plane of curvature, controlled by rotation of the inner sheath 26, or the extension of the inner sheath 26 into the uterus, or the extension of the needle 18 during injection, all of which provide additional degrees of position control.

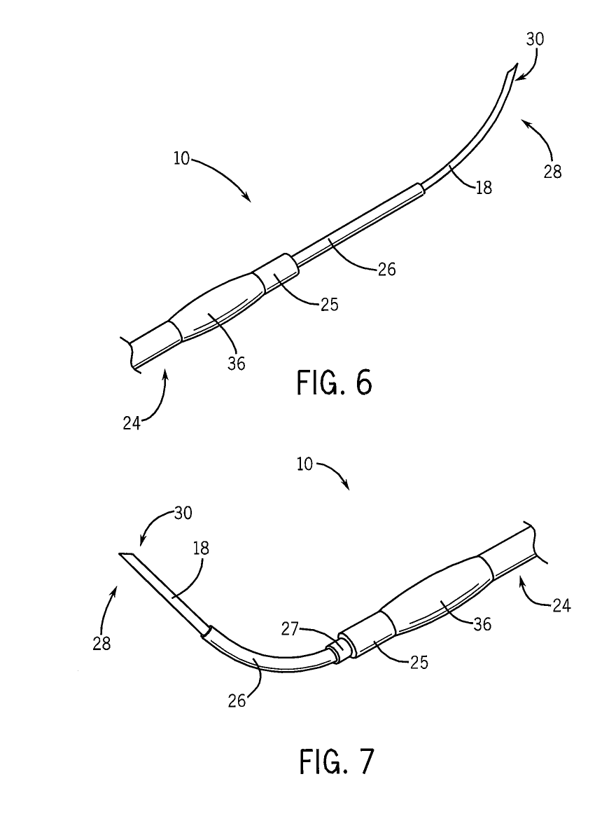

Turning now to FIGS. 6-7, and initially to FIG. 6, in a second embodiment of the present invention, the inner sheath 26 may be composed of a relatively rigid material similar to that of the outer sheath 25, and the needle 18 is preferably composed of a flexible material having a preformed curvature that is normally held straight by the combined guidance of the inner sheath 26 and the outer sheath 25. In operation of the second embodiment, the inner sheath 26 of the second embodiment of the present invention remains relatively rigid upon extension from the distal end 28 of catheter 10. Needle 18, however, is configured to automatically take on a predetermined curvature upon extension from inner sheath 26. Thus, the operator of the catheter 10 may manually adjust the curvature of the needle 18 to effectively steer a distal end 30 of the needle for injection of a fluid in the uterine cavity by controlling the extension of the needle 18 from inner sheath 26. In one variation on this embodiment, the needle 18 may be formed of a shape memory alloy such is Nitinol™. In this embodiment, the collar 40 and wire 38 are not used but simply the sliding of the needle 18 with respect to the collar 23 controls the amount of curvature.

Referring, now to FIG. 7, in a third embodiment of the present invention the inner sheath 26 is composed of a relatively flexible material that has a preformed curvature. A middle sheath 27 composed of a rigid material similar to that of the outer sheath 25 may fit between the inner sheath 26 and the outer sheath 25. The middle sheath 27 holds the inner sheath 26 in a straight configuration to a desired point of extension within the uterus. The inner sheath 26 may then be extended from the middle sheath 27 causing it to curve and providing a similar curvature to the flexible needle 18. As such, the third embodiment operates substantially similar to that of the second embodiment of the present invention permitting precise control of the needle 18 in curvature, extension and rotation (the latter formed by rotating the inner sheath 26 with respect to the outer sheath 25). As described above, the inner sheath 26 may be composed of a memory metal or alloy that is configured to assume a predetermined radius of curvature when exposed from the outer rigid sheath 25.

Referring now to FIG. 8, the catheter 10 of the present invention may be used for sentinel mapping of a tumor 46 attached to the uterine wall 48 below an opening to a fallopian tube 50. In a first step of this mapping process, plugs 52 may be inserted optionally into the fallopian tubes 50 to prevent liquid including possibly displaced cancer cells from exiting the fallopian tubes 50 into the abdomen. The design and placement of plugs 52 of this type are known in the area of contraception.

The catheter 10 may then be placed in the cervix 42 with the balloon 36 located at the neck of the cervix 42 with the inner sheath 26 and needle 18 retracted within the outer sheath 25. Liquid may then be introduced into the balloon 36 inflating it slightly to retain it and the outer sheath 25 in a fixed position and in a relatively leak-tight seal against the cervix 42. Liquid (not shown) may then be introduced in channel 35 between the inner sheath 26 and the needle 18 to slightly expand the uterine wall 48 to allow for maneuvering of the needle 18 preferably to a point where liquid is not expressed through the fallopian tubes 50 either as plugged or without plugging, in the latter case the liquid being contained by the natural restriction of the fallopian tubes 50 with limited distention of the uterine wall 48. Typically the amount of introduced fluid will be less than 200 mL.

In the embodiment of FIGS. 1-5 the inner sheath 26 may then be extended as shown in phantom, rotated if necessary and curved to direct the needle 18 to a first site 53 for the injection of a tracking medium. The needle may be retracted and the inner sheath straightened to maneuver beyond the tumor 46 to place contrast medium at first site 53. Additional target positions (not shown) may be placed about the periphery of the tumor 46.

A similar procedure may be used with the embodiments of FIGS. 6 and 7 by using a combination of rotation extension in curvature to place the needle 18 at the various desired locations. The liquid in the uterus and the metallic components of the catheter can allow visualization of this process using ultrasound.

Turning now to FIG. 9, a schematic representation of a portion of the lymphatic system 54 includes a plurality of lymph nodes 56 linked by a plurality of lymphatics 58 in a tree fashion leading from the tumor 46. Once the contrast media enters the lymphatic system near the tumor 46 it travels through the lymphatics 58, and the contrast media pools at the nodes near the tumor 46. Those lymph nodes 56 where the pooling first occurs are then identified as the sentinel lymph nodes and are removed, thereby determining whether the cancer has spread and further preventing the spread of the cancer from the uterus through the lymphatic system 54.

Although the best mode contemplated by the inventors of carrying out the present invention is disclosed above, practice of the present invention is not limited thereto. It will be manifest that various additions, modifications and rearrangements of the aspects and features of the present invention may be made in addition to those described above without deviating from the spirit and scope of the underlying inventive concept. The scope of some of these changes is discussed above. The scope of other changes to the described embodiments that fall within the present invention but that are not specifically discussed above will become apparent from the appended claims and other attachments.

We claim:

1. A steerable sonohysterographic injection catheter comprising:
a tubular shaft extending along an axis and having a proximal end and a distal end;
a needle receivable in the tubular shaft and extendible therefrom, the needle sized for intrauterine insertion to deliver a fluid to a tumor location in a patient's uterus; and
a sealing device disposed on the catheter and configured for placement at an opening of patient's cervix, the sealing device configured to prevent the fluid from leaking out of the patient's uterus through the patient's cervix;
wherein a distal end of the needle may be controllably curved with respect to the axis within the uterus so as to allow the distal end of the needle to be steered to different locations about the tumor.

2. The catheter of claim 1 wherein the tubular shaft comprises a rigid outer sheath and a flexible inner sheath, the inner sheath being extendible from the outer sheath.

3. The catheter of claim 2 further comprising a control member operably coupled to a distal end of the inner sheath and configured to displace a side of the inner sheath thereby causing the inner sheath to curve, wherein the curving of the inner sheath effectively curves the needle.

4. The catheter of claim 3 wherein the control member comprises a filament having a proximal end and a distal end, wherein the proximal end of the filament is configured to be actuated by a user to pull the side of the inner sheath.

5. The catheter of claim 3 further comprising a handle coupled to a proximal end of the tubular shaft and in communication with the control member for control thereof.

6. The catheter of claim 5 wherein the handle includes a first port configured to receive the needle therethrough for positioning the needle within the tubular shaft, wherein a proximal end of the needle is adapted to be coupled to a syringe for delivery of the fluid to the needle.

7. The catheter of claim 6 wherein the sealing device comprises a balloon and the handle further comprises a second port in communication with the balloon, wherein the second port is configured to deliver an inflation liquid to the balloon for selective inflation thereof.

8. The catheter of claim 1 wherein the needle is substantially flexible and configured to assume a predetermined curvature upon extension from the tubular shaft.

9. The catheter of claim 8 wherein the needle is composed of a memory alloy.

10. The catheter of claim 9 wherein the memory alloy is nickel titanium.

11. The catheter of claim 2 wherein the inner sheath is configured to assume a preferred radius of curvature upon extension from the outer sheath.

12. The catheter of claim 11 wherein the needle is flexible to follow the preferred radius of curvature of the inner sheath upon extension from the inner sheath.

13. The catheter of claim 11 wherein the inner sheath comprises a shape memory alloy configured to automatically assume a predetermined shape.

14. The catheter of claim 13 wherein the flexible memory metal is nickel titanium.

15. A method of identifying sentinel nodes associated with uterine cancer comprising the steps of:
(a) inserting a catheter into a uterus;
(b) introducing a liquid through the catheter to separate the uterine walls while substantially preserving closure of the fallopian tubes;
(c) steering a distal end of the catheter at a point about a tumor; and
(d) inserting a hollow needle through the catheter to be guided by the catheter to the point for injection of a tracking material in tissue about the tumor to identify a sentinel node.

16. The method of claim 15 wherein the catheter provides an outer inflatable balloon for sealing an opening of the uterus.

17. The method of claim 15 wherein the distal end of the catheter may be steered to different angles with respect to a principal axis of the catheter.

18. The method of claim 15 further including the step of inserting plugs into the fallopian tubes to prevent the flow of liquid into the fallopian tubes.

19. The method of claim 15 wherein an amount of liquid used to separate the uterine walls is less than 200 mL.

20. The method of claim 15 further comprising the step of imaging the uterus to identify a location of the tumor.
